(19) United States
(12) Patent Application Publication
(10) Pub. No.: US 2006/0111767 A1
(43) Pub. Date: May 25, 2006

(54) Medical implant systems

(75) Inventors: Donald R. Olson, Dundee, OR (US); David W. Altman, Dundee, OR (US)

Correspondence Address: LUEDEKA, NEELY & GRAHAM, P.C., P O BOX 1871, KNOXVILLE, TN, 37901, US

(73) Assignee: Medi-Screw, Inc.

(21) Appl. No.: 11/314,836

(22) Filed: Dec. 21, 2005

Related U.S. Application Data
(63) Continuation of application No. 10/854,431, filed on May 26, 2004.
(60) Provisional application No. 60/474,437, filed on May 30, 2003.
(60) Provisional application No. 60/563,539, filed on Apr. 19, 2004.
(60) Provisional application No. 60/570,961, filed on May 13, 2004.

(51) Int. Cl. A61N 1/05

(52) U.S. Cl. 607/116

(57) ABSTRACT

Medical implant systems for a variety of medical uses including stimulating tissue, locating nerves during a medical procedure, measuring nerve activity, stimulating bone growth, assisting hearing, providing eyesight, delivering medicaments, and facilitating positioning and tracking of positions of medical implants.

### CROSS-REFERENCE TO RELATED APPLICATIONS

This application claims priority to:

U.S. Provisional Application Ser. No. 60/474,437, filed May 30, 2003, and entitled MEDICAL FASTENER APPLICATION SYSTEM.

U.S. Provisional Application Ser. No. 60/563,539, filed Apr. 19, 2004, and entitled MEDICAMENT DELIVERY SYSTEM.

U.S. Provisional Application Ser. No. 60/570,961, filed May 13, 2004, and entitled POSITIONING SYSTEM.

### FIELD OF THE INVENTION

This invention relates generally to medical devices and systems for installing implantable medical workpieces, and to methods for using the same. Devices and systems according to the various aspects of the invention may be used for a variety of medical applications and may be used for identifying the presence of nerves and/or to stimulate nerves and other tissue, to deliver treatment agents, to provide positional information about the location of a fastener or other implant such as an artificial spinal disk, and information about the condition of an implant site.

### BACKGROUND AND SUMMARY OF THE INVENTION

The invention advantageous enhances minimally invasive surgical devices and procedures, particularly those utilizing medical implants, especially medical fasteners.

In this regard, improved methods and apparatus are provided in the field of medical fasteners, implants, and other workpieces. Such methods and apparatus may be suitable for a number of applications, such as for nerve or other tissue detection during spinal surgical procedures. For example, during installation of medical fasteners, such as spinal screws, it is important to avoid disruption of nerves. Other applications include tissue stimulation, pain modulation, nerve manipulation, and the like wherein a nerve or other tissue is stimulated to achieve a desired result.

The methods and apparatus may also be used to facilitate the delivery of medicaments, such as pain relief medicaments, to enhance the effectiveness of the medicaments.

The methods and apparatus may also be used in conjunction with surgical devices and methods that involve the use of images generated by imaging systems such as computer tomographic imagers (CT), magnetic resonance imaging (MRI), and the like. In this regard, the invention enables information about the position and orientation of medical workpieces to be obtained to facilitate desired placement of medical workpieces, such as fasteners and implants, and subsequent return to such workpieces.

In one preferred embodiment, a medical fastener system is provided which includes a fastener, such as a screw, and a driver for installing the fastener within a patient. The driver and fastener are each configured to have communicating bores, preferably substantially co-linear bores extending through their lengths, for passage of an electrically communicative element, such that the electrically communicative element is exposed to and in electrical communication with the exterior of the fastener. A source of electrical power and a detector are each in electrical communication with the electrically conductive element.

When an electrical force is applied to the electrically communicative element via the source of electrical power, electrical signals will be generated by the patient if electrically responsive tissue, such as a nerve, is proximate the fastener. The patient generated signals are conducted via the fastener and the electrically communicative element to the detector to indicate that electrically responsive tissue, such as a nerve, is proximate the fastener. Thus, the surgeon may be alerted to the presence of such tissue and take appropriate action.

In another aspect of the invention, the driver and fastener are each configured to have communicating bores for passage of a tissue stimulating device, such that the stimulating device is exposed to and in communication with the exterior of the fastener. A source of stimulating power and a detector are each in communication with the stimulating device. When a stimulating force is applied to the stimulating device, signals, such as signals corresponding to neuro-muscular responses will be generated by the patient if electrically responsive tissue, such as a nerve, is proximate the fastener. The patient generated signals are communicated to the detector to indicate that electrically responsive tissue is proximate the fastener.

In another aspect, fasteners are provided and used for pain modulation.

In a further aspect, fasteners according to the invention and configured for enabling tissue stimulation may be operatively associated with somatosensory devices, such as receivers, and desirably located for sensing somatosensory evoked potentials.

In a still further aspect, fasteners according to the invention may be configured for providing electrical stimulation to augment bone growth or bone regrowth.

In another aspect, fasteners according to the invention may be used to provide an artificial eye, wherein the fastener is located to stimulate optical tissue, with electrical signals generated by a camera transmitted to the fastener.

In still another embodiment, the invention relates to a stimulating device which includes a light detector, such as a photodiode, located adjacent a nerve or tissue to be stimulated and a light emitter, such as a light emitting diode, located remote from the photodiode and in optical communication with the photodiode. The device may, in one example, be configured for assisting hearing and/or sound amplification, wherein the photodiode detects light generated by the light emitting diode and generates a corresponding electrical signal to stimulate tissue within the ear canal.

In another aspect, a preferred medicament delivery system includes a screw having a bore defined therethrough to define an inlet for introducing a medicament into the bore and an outlet in flow communication with the inlet for passage of the medicament from the bore for introduction to a desired location within a patient. A seal is operatively associated with the inlet for selectively sealing and unsealing the inlet.

A further aspect relates to an artificial spinal disc system which includes an artificial spinal disc having a transmitter coupled to an electronic position sensor for generating electronic signals corresponding to the position and orientation of the electronic position sensor in three dimensional space.

Another aspect of the invention provides a positioning system that provides improved positional information of a workpiece, such as a medical fastener, artificial spinal disc, or other implantable medical device. The positioning system preferably includes a rotatable workpiece having a head thereon. The head defines a plurality of reference structures located thereon and oriented to define a plurality of sets of the reference structures, with the reference structures of each set being arranged in a common plane, but with each set of reference structures being located in distinct but parallel planes. A driver is provided having a plurality of drive surfaces located and configured to abut the reference structures when the driver and the head are mated.

First sensors are located on the reference structures and second sensors are located on the drive surfaces. Each of the first sensors are operable with one of the second sensors to enable electrical signals to be generated corresponding to signals transmitted and received therebetween. A computer control circuit is in electrical communication with one of the first or second set of sensors for receiving the generated electrical signals.

The positioning system may further facilitate docking of a medical driver to a previously installed medical fastener for subsequent medical procedures or removal thereof. This is particularly advantageous when attempting to return to previously implanted fasteners which have been in the patient for an extended period of time and have become overgrown with tissue. The positioning system may also preferably be incorporated into the artificial spinal disc system.

### BRIEF DESCRIPTION OF THE DRAWINGS

Further features of preferred embodiments of the invention will become apparent by reference to the detailed description of preferred embodiments when considered in conjunction with the figures, which are not to scale, wherein like reference numbers, indicate like elements through the several views, and wherein,

FIGS. 1A and 1B are exploded and assembled views, respectively, showing an embodiment of the invention wherein a medical fastener and a driver for installing the fastener are each configured to have bores extending through their lengths for passage of an electrically communicative element.

FIGS. 2A-2C shows castellated screw and driver configurations of the type preferably incorporated into the screw and driver of the invention.

FIG. 3 shows an alternate embodiment of a medical fastener in accordance with the invention.

FIGS. 4A-4C show alternate embodiments of systems for stimulating nerve tissue.

FIG. 5 shows a medical fastener of FIG. 1 electrically connected to a receiver for sensing somatosensory evoked potentials.

FIG. 6 shows an alternate embodiment of a medical fastener and driver which utilizes a photodiode for stimulating nerves.

FIG. 7 shows an alterative application of a photodiode system utilized within an ear for assisting hearing.

FIG. 8 is an exploded side view of a medicament delivery system in accordance with a preferred embodiment of the invention.

FIG. 9 is a top plan view taken along line 9-9 of FIG. 8.

FIG. 10 is a top plan view taken along line 10-10 of FIG. 8.

FIG. 11 shows a delivery system according to the invention which incorporates an internal pump.

FIG. 12 is a side view of components of a positioning system in accordance with a preferred embodiment of the invention.

FIG. 13 shows a screw component of the system of FIG. 12.

FIG. 14 is a top plan view showing reference points associated with the head of the screw of FIG. 13.

### DETAILED DESCRIPTION

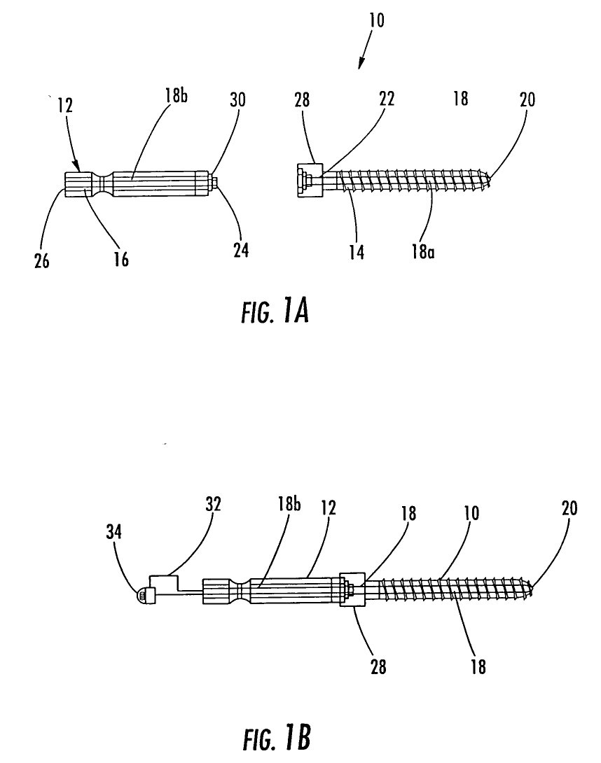

FIGS. 1-7

The invention relates in one aspect to medical implant systems. In various embodiments, the preferred implant is a medical fastener, such as a medical screw. The fastener systems advantageously enable detection of nerves during installation of medical fasteners and stimulation of tissue for various medical purposes. The invention is described in connection with preferred embodiments primarily involving the stimulation or detection of nerve tissue. However, it will be understood that essentially most body tissue including blood vessels and ligaments, is at least to some degree electrically responsive and may be interfaced with the methods and apparatus of the invention.

In one embodiment a system is configured for spinal surgeries wherein a screw is installed in spinal bone by use of a driver. In this regard, and with reference to FIGS. 1A and 1B, a screw 10 and a driver 12 are each configured to have bores, preferably co-linear bores 14 and 16, respectively, extending through their lengths for passage of an electrically communicative element 18, such as an electrode wire. Thus, the screw 10 includes apertures 20 and 22 at the respective ends thereof, and driver 12 includes apertures 24 and 26 at the respective ends thereof. The electrically communicative element 18 serves as a tissue stimulating device, and preferably a nerve stimulating device.

As will be appreciated, the electrically communicative element 18 need not be continuous, e.g., one-piece, but may have multiple pieces electrically coupled together. For example, the screw 10 may incorporate a first segment 18a of wire and the driver 12 a second segment 18b, with the first and second segments 18a and 18b electrically coupled as by mating contacts located at the respective apertures 22 and 24 when the driver is placed on the screw 10.

The electrically communicative element 18 is exposed to and in electrical communication with the exterior of the screw 10 via the aperture 20 of the bore 14 at the tip of the screw 10. For this configuration, the screw 10 is preferably made of a metal. However, the screw 10 may be made of a non-electrically conductive material, such as plastic or a re-absorbable material, having electrically conductive elements or coating materials thereon.

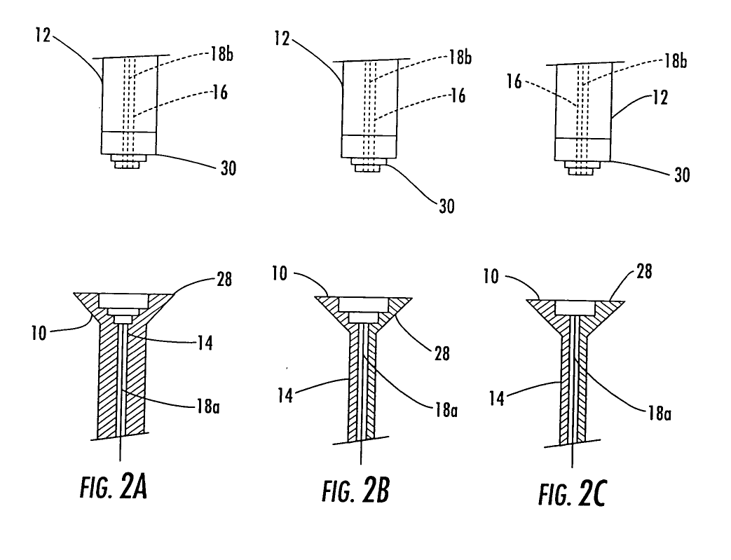

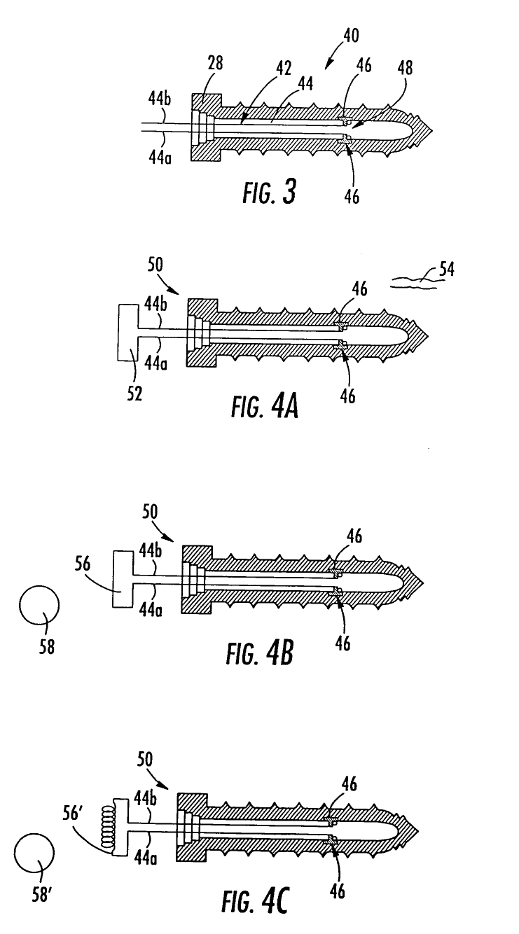

Head 28 of the screw 10 and interfitting tip 30 of the driver 12 are preferably configured in the manner of the male/female castellated screw head and driver configurations of the screws and drivers available from Uni-Screw Worldwide, Inc., of Knoxville, Tenn., and described in U.S. Published Application No. 2003/00538887, published Mar. 20, 2003, and entitled “Screw Head Formation,” and U.S. Published Application No. 2003/0075027, published Apr. 24, 2003, and entitled “Driving Heads For Fasteners,” incorporated herein by reference in their entireties. Examples of such head and driver configurations are the castellated configurations shown in FIGS. 2A, 2B, and 2C. As will be noted, the screw head provides a female structure and the driver 12 provides a male structure. It will be appreciated that the driver and screw head may be of opposite configuration, such that the driver represents the female structure and the screw head represents the male structure.

An electrical force is applied to the electrically communicative element. The electrical force preferably corresponds to electrical forces of the type commonly used for various medical procedures, including direct current and alternating current as may be appropriate, with such force most preferably being applied in pulses for the examples described herein. If a nerve or other electrically responsive tissue is proximate the screw 10, it will be stimulated by the electrical force and electrical signals will be generated in response.

For example, in the case of nerve tissue, electrical signals corresponding to neuro-muscular (EMG) responses will be generated by the patient. These patient generated electrical signals will be conducted via the screw 10 and the electrically communicative element 18 to a detector and circuitry 32 associated with the driver 12 and in electrical communication with the electrically communicative element 18 to indicate that a nerve is proximate the screw. For example, a signal device 34, such as an audible alarm may sound or a light, e.g. a light emitting diode or the like, may be illuminated. If desired, an amplifier may be included to boost the signal to a level sufficient to operate a desired signal device.

The electrical signals may also be used to determine other information about the implant site, such as determining the size of a cavity in the bone, which is particularly useful information when performing spinal surgeries. In regard to cavities, it will be appreciated that bone and bone marrow have different conductivities and will therefore provide different electrical signals when stimulated. In this manner, the screw 10 may be manipulated in an area of interest to determine the extent of the cavity, based on the electrical signals generated. This information may be used to assess the size of the cavity and in positioning workpieces such as implants and medical tools during a procedure.

FIG. 3 shows an alternate embodiment of a screw 40 for use with the driver 12. The screw 20 includes a blind bore 42 for receiving electrically conductive element 44. The electrically conductive element 44 is preferably provided as by a pair of wires 44a and 44b. The wires 44a and 44b may terminate at the head of the screw 40 and connect to an electrical contact, such as may be contacted by a similar contact on the driver 12, or may extend past the head end to electrically couple with another device, as described more fully below.

One or more electrical contacts 46 are located within the bore 42, preferably adjacent a blind or closed end 48 of the bore 42. The contacts 46 are in electrical communication with the electrically conductive element 44 and extend to an exterior portion of the screw 40. This configuration is particularly suitable for use with plastic screws, bio-absorbable screw, and the like which are generally not electrically conductive. The screw 40 preferably includes a similar head configuration as the screw 10, thus incorporating the head 28, and may be used with the driver 12 in the manner described in connection with the screw 10.

In yet another aspect of the invention, fasteners according to the invention may be utilized to stimulate tissue for therapeutic purposes. In one aspect, the stimulation may be supplied for providing pain relief. In other aspects, the fasteners may serve as electrodes for application of low frequency alternating current for stimulating bone growth or regrowth. For such applications, the current is preferably induced as by an external low frequency electromagnetic field.

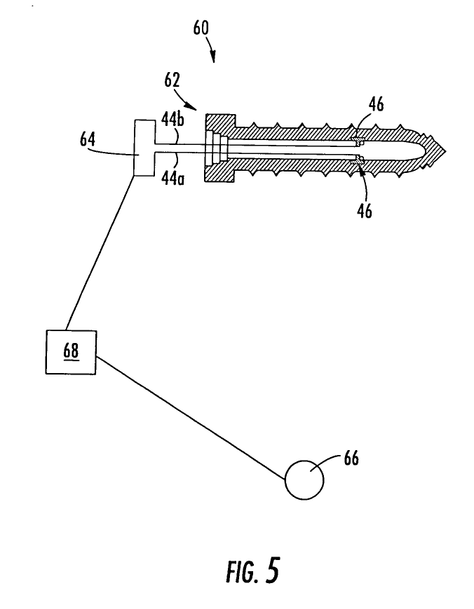

For example, with reference to FIG. 4A, a fastener 50, preferably corresponding in structure to the screws 10 or 40 described previously, may be directly electrically coupled to a source of electrical power 52, and positioned adjacent to electrically responsive tissue, such as a selected nerve or nerve bundle of a patient. As noted previously, while many of the preferred embodiments are described for use in context of stimulating nerve tissue, it will be understood that the invention is not limited to use in connection with nerve tissue and is applicable to essentially most body tissue.

The source of electrical power 52 is preferably a battery-operated power supply implanted within the body adjacent the fastener and electrically coupled thereto for delivering low power pulses. Depending upon space limitations, the source of electrical power 52 may also preferably be located within the bore of the fastener 40. As shown, the fastener 50 is configured as the screw 40, such that electrical power is conducted via the wires 44a and 44b to the contacts 46 for stimulating nerve tissue 54 proximate the fastener 50.

With reference to FIG. 4B, electrical energy may be remotely coupled to the fastener 50, wherein the electrical source 52 is replaced with an RF receiver 56. While the receiver 56 is shown located away from the channel or bore of the fastener 50 for ease of representation, it will be understood that the receiver 56 may also preferably be located within the channel or bore of the fastener 50. An external RF transmitter 58 may be provided remote from the receiver 56 for transmitting radio frequency energy to the receiver 56. The energy received by the receiver 56 is then conducted via the wires 44a and 44b to the contacts 46 for stimulating tissue. It will be appreciated that the screw 10 described above may similarly be electrically connected to an RF receiver for receiving energy from an RF transmitter.

The embodiment of FIG. 4B is believed to be particularly suitable for providing retinal fasteners wherein the transmitter 58 is coupled to a camera, such as may be located on the eyeglasses of a user and the fastener 50 is configured to have a length of about 1 mm for installation into the retina of a patient. As will be appreciated, electrical signals from the camera are transmitted to the fastener for stimulating optical nerves for providing sight, thus providing an artificial eye. In this regard, it is noted that the transmitting components of each of the various embodiments described herein may each be couplable to a variety of signal generating devices such as digital cameras which may function to generate electrical signals that may be transmitted or otherwise communicated from the transmitting component to the receiver component for effecting tissue stimulation.

In a particularly preferred embodiment, the RF receiver 56 of FIG. 4B may preferably be provided by a radio frequency microstimulator device available under the tradename BION from Advanced Bionics Corporation.

The receiver 56 and transmitter 58 may also be reversed, with the transmitter 58 located within the fastener (or other workpiece). Also, the receiver 56 and transmitter 58 may be combined in the manner of a transceiver. In circumstances wherein the fastener, screw or other workpiece is configured to have a transceiver or transmitter therein, a sensor or other information generating device is placed in electrical communication with the transceiver or transmitter for providing electrical signals corresponding to the desired information to be gathered and transmitted. For example, an RF or other wireless implant may be provided by providing a glucose sensor, pressure sensor, temperature sensor, or other sensor device in conjunction with a transmitter or transceiver for generating and transmitting information corresponding to the bodily conditions. In addition, it will be understood that sensors may be provided for outputting signals that are not dependent upon contact with bodily fluids or other environmental conditions, such as information corresponding to location in three-dimensional space.

In this regard, it will further be understood that while a wireless receiver or transmitter or transceiver may preferably be used in conjunction with the contacts 46 (or electrically communicative element 18) for applications involving electrical stimulation of tissue, the wireless devices and associated sensors may also preferably be provided within the bore of the fastener 50 (or other such configured workpiece) absent the contacts 46 or in the bore of the screw 10 (or other such configured workpiece) absent the electrically communicative element 18 for measuring and generating parameters that do not involve stimulation of tissue, such as for determining pressure or providing positional information. Finally, it will be appreciated that more than one wireless device and sensor may be provided, or that the provided wireless device be capable of generating a variety of signals corresponding to position or environmental aspects.

With reference to FIG. 4C, as an example of another type of wireless device, the RF receiver is replaced with a conventional inductance coil 56′ and the RF transmitter 58 is replaced with a conventional source 58′ of a low frequency electromagnetic field for inducing a current in the coil. As will be appreciated, the locations of the transmitter and receiver may be reversed.

Fasteners or other workpieces according to the invention may also be operatively associated with somatosensory devices, such as receivers, and desirably located for sensing somatosensory evoked potentials for diagnosing tissue damage, such as nerve damage, and for monitoring purposes during spinal surgery.

In this regard, it has been observed that monitoring of nerve signal speed may be useful for diagnosing nerve damage in some cases. Somatosensory evoked potentials are recorded or otherwise determined to assess the speed at which nerves are conducting electrical signals across the spinal cord. For example, if the spinal cord is pinched, the signals will generally travel slower than for an unpinched spinal cord. The invention advantageously enables location of a detection device at one or more desired locations along the spine for monitoring nerve signal speed.

With reference to FIG. 5, there is shown a detector system 60 in accordance with a preferred embodiment for detecting somatosensory evoked potentials. The detector system 60 includes a fastener 62 electrically coupled to a conventional receiver 64, such as is commonly used in conjunction with detectors for monitoring somatosensory evoked potentials. The fastener 62 preferably substantially corresponds to the fasteners 10 and 40, described previously. In this regard, the fastener 62 as shown corresponds to the fastener 40 and includes the wires 44a and 44b and contact pads 46.

In monitoring nerve speed, for example, one or more of the fastener 62/receiver 64 combinations is threadably received along the spine proximate a desired nerve. This is preferably accomplished in the manner previously described for installing the fastener 10, with installation of the fastener portion of the detector ceased at the first indication of nerve proximity. Following this, electrical stimulating pulses are preferably supplied to a remote region of interest as electrical stimulating pulses supplied by a conventional electronic stimulator 66, to a portion of the foot of the patient. A computer controller 68 operatively associated with the stimulator 66 and the receiver 64 may be used to measure time lapse between application of the stimulating pulses and detection thereof by the receiver 64.

In a similar manner, the fastener 62 may be used as an electrode for electrophysiological monitoring of the spinal cord during spinal surgery.

Accordingly, the term “tissue stimulating device” as used herein, will be understood to refer to electrically communicative devices through which electrical power can travel to apply electrical force toward tissue to directly stimulate the tissue or through which electrical power as may be generated by tissue may travel for detection. In the context of the preferred embodiments, therefore, the term “nerve stimulating device” will be understood to a tissue stimulating device for stimulating nerve tissue, it being understood that various body tissue may be stimulated according to the invention.

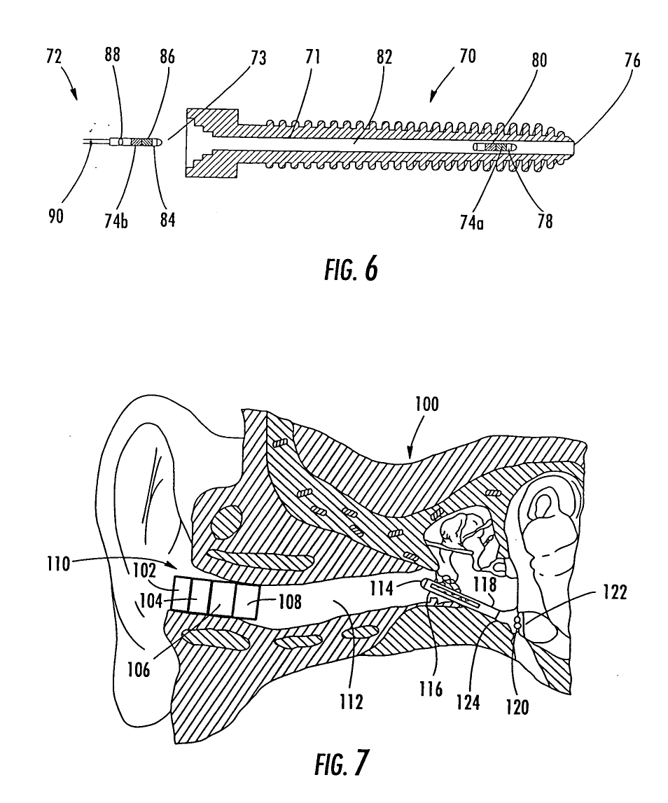

Turning now to FIG. 6, there is shown an alternate embodiment of a fastener application system that incorporates a light emitting/light detection system that preferably utilizes a photodiode or a photoresistor for detecting light and generating an electrical signal in response that may be used to stimulate tissue.

In a preferred embodiment, a system is provided having a fastener, such as a screw 70, and a driver 72 for installing the screw 70 within a patient, such as during spinal surgery. The head of the screw 70 and the interfitting tip of the driver 72 are preferably configured as described previously for the screw 10 and driver 12, most preferably corresponding to the configurations of FIGS. 2A-2C.

Screw 70 and driver 72 are each configured to have bores, preferably co-linear bores 71 and 73, respectively, extending through their lengths. The screw 70 and driver 72 incorporate assemblies 74a and 74b of a light emission/detection system such as a photodiode system located within the bores 71 and 72, respectively, for generating light, detecting light, and generating an electrical signal corresponding to the detected light for stimulating nerves or other tissue and detecting stimulated tissue during a medical procedure.

The bore 71 preferably extends the length of the screw 70 and has an open terminal end 76. Assembly 74a preferably includes a photodiode 78 located within the bore 71 adjacent the end 76 for stimulating nerves or other tissue. Alternatively, the bore 71 may be a blind bore, with one or more photodiodes, such as the diode 78, extending through apertures defined through the sidewall of the bore 71 to the exterior of the screw 70. An aspherical lens 80 is preferably adjacent the diode 78, and a optical fiber 82 within the bores 71 and 73 enables communication between assembly 74b of the photodiode system, preferably located within the bore 73 of the driver 72.

The assembly 74b of the driver 72 preferably includes a laser light emitting diode 84, an amplifier 86, a rotary optical modulator 88 for modulating the amplitude impulse, preferably a spinning aspherical lens, and an optical fiber 90. The fiber 90 can communicate with a detector device, such as signal device 34 described previously, for indicating proximity of tissue, such as a nerve and supplying other communication requirements.

FIG. 7 shows an alterative application of a light emission/light detection system 100, preferably a photodiode system utilized within an ear for assisting hearing by stimulating tissue. The system 100 preferably utilizes a microphone 102, a receiver 104, and amplifier or processor 106, and a laser emitting diode 108 adjacent ear opening 110 of an ear canal 112. Located deeper within the ear canal 112 adjacent the tympanic membrane of the ear is an aspherical lens 114, an optical fiber 116, a photodiode 118, and electrodes 120. These components are positioned adjacent a round window 122 of the ear canal structure. The positioning may be accomplished as by locating the components within a suction tube 124, which may be detachably positioned within the ear canal.

In operation, it will be understood that sound waves are received via the microphone 102 and receiver 104, amplified by the amplifier 106, and transformed into light by the light emitting diode. The light is focused and/or modulated by the lens 114 and transmitted via the optical fiber 116 to the photodiode 118. The photodiode 118 converts the light to electrical signals which are conducted via the electrodes 120 to stimulate the round window 122 of the ear canal structure and thereby assist hearing.

The workpieces have been described herein generally in terms of fasteners, preferably medical screws, and in connection with stimulating or otherwise electrically interacting with tissue to generate a signal to obtain information about the tissue. However, it will be understood that the workpieces may of other configuration, including in the form of medical implants such as artificial spinal discs, with the implant including a head corresponding to the head of the screw. For example, artificial spinal discs may be configured in the manner of the screws 40, 50, 62, and 70, including a head for mating with a driver and including the described electrically conductive elements or wireless devices such as RF receivers, transmitters, transceivers and any associated sensors. As described previously, the workpiece, whether a fastener or spinal disc or other workpiece, may include one or more wireless devices having capabilities such as stimulating tissue, measuring environmental conditions such as temperature, pressure and the like, and providing positional information.

It is particularly preferred to have a workpiece configured as an artificial spinal disc. In such embodiment, a conventional artificial spinal disc may be modified to include a head, preferably the castellated head described herein in connection with the fasteners, with a wireless device configured to provide three dimensional positional information, e.g., such as a transmitter or transceiver in electrical communication with a sensor capable of generating signals corresponding to its position and orientation in three-dimensional space. As the position and orientation of the position sensor is fixed and known relative to the dimensions of the artificial spinal disk, the positional information will be related to the position and orientation of the disk in the body. Likewise, the disc also preferably incorporates electrically stimulating structure, such as the electrically communicative element 18 or the contacts 46 in electrical communication with the transceiver, for stimulating tissue in the spine for determining the dimensions of the cavity into which the spinal disc is to be installed.

Such a configured workpiece is particularly advantageous in the form of a spinal disc in that the position of the disc may be manipulated to observe the dimensions of the cavity of the spine into which it is to be installed, as well as facilitating precise positioning of the disc utilizing the positional information transmitted from the disc.

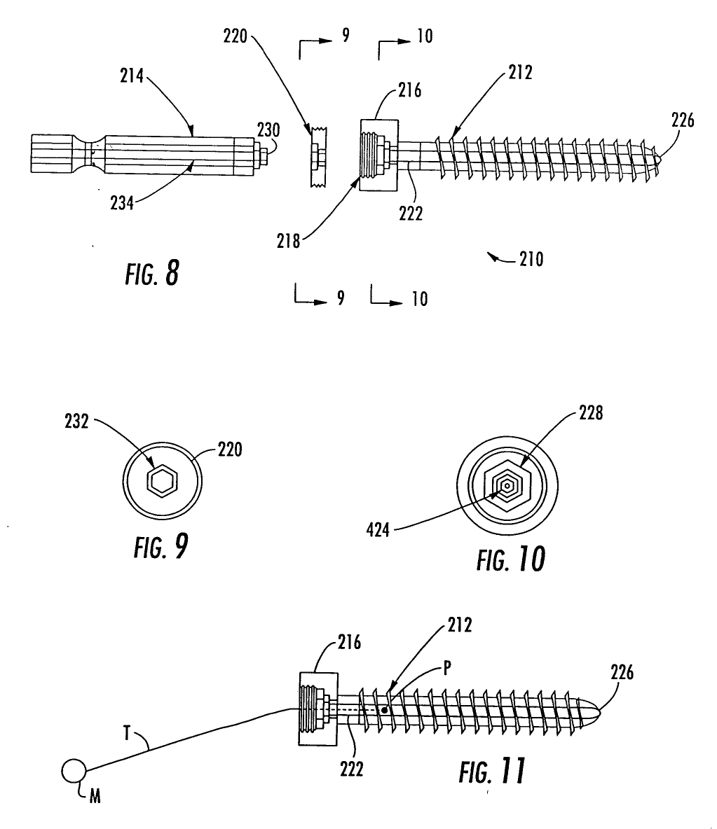

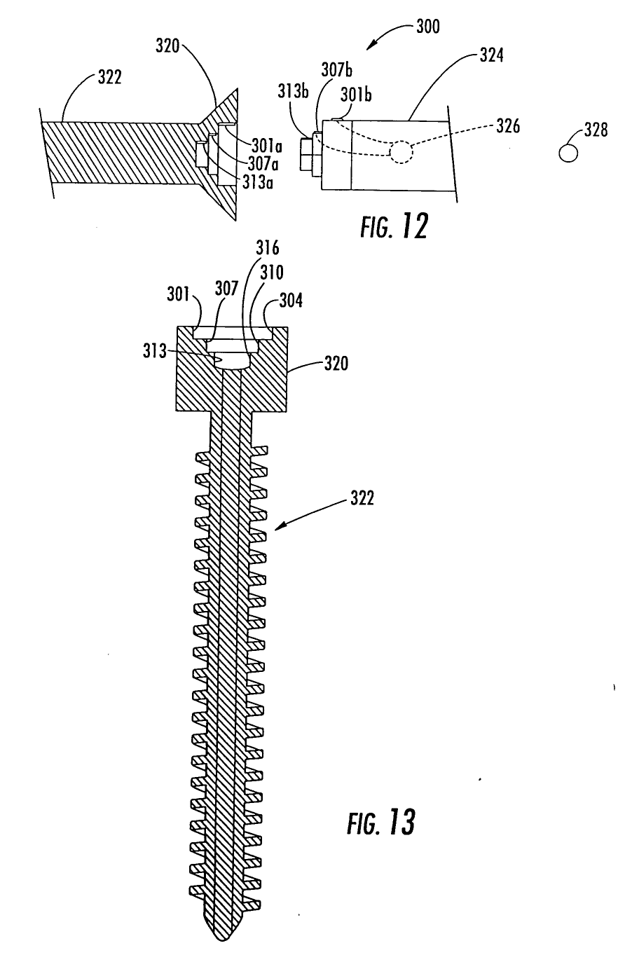

FIGS. 8-11

With reference to FIGS. 8-11, there is seen a delivery system 210 in accordance with an alternate preferred embodiment of the invention. The system 210 includes a screw 212 which may preferably be positioned at a desired location within the body of a patient as by use of a driver 214.

The screw 212 includes a head 216 configured to define a threaded socket 218 for threadably receiving a threaded cap 220. A bore 222 preferably extends through the center of the screw 212, with the bore 222 being open at the two ends thereof for providing a flow conduit for delivery medicament. For example, aperture 224 is located at the otherwise blind end of the socket 218 for introduction of medicament into the bore 222 and a tip end 226 of the screw 212 is open to define an exit location for the delivery of medicament into the patient. Alternatively, the exit location may be through a sidewall of the screw 212. For delivery of pain medication to nerve tissue, the bore preferably has a diameter of about 0.0625 inches.

The tip end 226 and the threads of the screw 212 may have a variety of configurations depending upon the desired location in the body to be targeted for drug delivery. For example, the screw 212 may be used for delivery of supplying medicament to tissue such as nerve tissue, bone marrow, ligaments, or a blood flow source such as veins or arteries. In the case of veins or arteries, it is preferred that the tip end 226 be configured to fixedly receive a needle or other pointed cannula.

The cap 220 may be threadably received or removed from the socket 218 for desirably sealing or unsealing access to the bore 222 via the socket 218. As will be appreciated, the cap 220 and the manner in which it interfits with the socket 218 is selected to enable a substantial seal against passage of fluid to or from the bore 222 via the aperture 224 when the cap 220 is fully seated within the socket 218.

The screw 212, cap 220, and the driver 214 preferably utilize a male/female castellated configuration of the type incorporated into the screws and drivers available from Uni-Screw Worldwide, Inc., of Knoxville, Tenn. For example, a castellated drive surface 228 is preferably defined within the socket 18 for receiving an interfitting castellated driver surface 230 defined on the tip of the driver 214. Likewise, a castellated drive surface 232 is also provided on the cap 220 for receiving the driver surface 230 of the driver 214. The driver 214 fits the surface 228 of the screw 212 for installation/removal of the screw 212, and the surface 230 of the cap 220 for installation/removal of the cap 220 from the socket 18. Thus, the threads for the cap 220 are preferably fine threads which have a small resistance to facilitate removal/installation of the cap 220 without causing movement of the screw 212 during manipulation of the cap 220. It will be appreciated that the driver and screw head may be of opposite configuration, such that the driver represents the female structure and the cap and the screw head represent the male structure.

The screw 212 may be inserted, as by use of the driver 214, to a desired location within the body of the patient for delivery of medicament. For example, the screw 212, with the cap 220 removed, may be located proximate nerve tissue suspected to be causing the patient to experience pain. In this regard, it is preferred that the screw 212 and the driver 214 be further configured in the manner of the screw 10 (or screw 40) and the driver 12, for example, to include nerve sensory elements, such as the electrically communicative element 18 and the detector and circuitry 32 to facilitate desired placement of the screw and/or nerve proximity information. The screw 212 and the driver 214 may also be configured to include a light emitting/light detection system such as described herein in connection with FIG. 6 for facilitating desired placement.

To facilitate application of a medicament, the bore 222 may be initially charged with a desired medicament, such as a pain relief medicament, e.g., narcotic, anesthetic, or other medication, drug, or treatment agent. Alternatively, the bore 222 may be charged with a medicament after installation of the screw 212. This may be accomplished on a continual or periodic basis.

After the bore 222 of the screw 212 is desirably charged with medicament, the cap 220 may be installed as by use of the driver 214 to serve as a seal between introduction of medicament to the bore 222. After treatment is accomplished, the screw 212 is preferably removed from the patient as by use of the driver 214.

A medicament supply conduit may be threadably received by the socket 218. The supply conduit may extend to a location exterior the body of the patient and connected to a source of medicament. Alternatively, the supply conduit and an implantable source of medicament may be located within the body. In addition, a pumping device, such as a micro or nano pump P, may be located in the bore 222 of the screw 212 for supplying medicament via the bore 222, with the pump in flow communication with a source of medicament M via the supply conduit such as tubing T or the like.

Alternatively, the driver 214 may be utilized to charge the bore 222. In this regard, the driver 212 preferably includes a bore 234 extending therethrough which may serve as a conduit for delivering medicament to the screw 212. For example, the bore 234 may extend through the driver surface 230 such that the bore 234 and the bore 222 are placed in flow communication with one another when the driver surface 230 is inserted into the drive surface 228 of the screw 212. Medicament may be introduced into the bore 234 via the open end of the bore 234 opposite the drive surface 230, or the open end of the bore 234 may be placed in flow communication with a source of medicament.

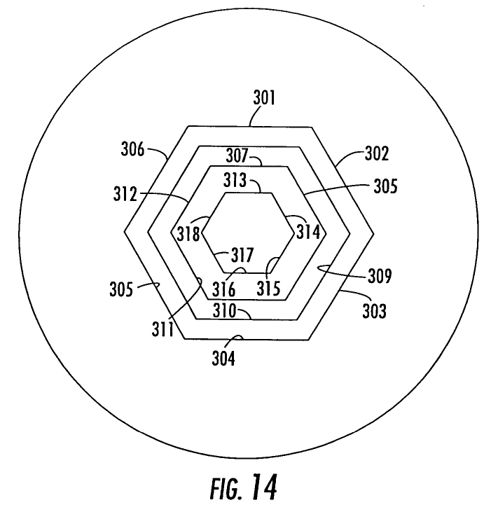

FIGS. 12-14

With reference to FIGS. 12-14, the invention relates to a positioning system 300 that is particularly suitable for positioning screws and for enabling positional information about the location and orientation to be gathered. The system 300 may preferably be incorporated into the various embodiments described herein in connection with FIGS. 1-11 and will be understood to be applicable to other workpieces besides the described screws. In this regard, the positioning system 300 is believed to be particularly suitable for facilitating installation and removal of retinal fasteners such as described previously in connection with FIG. 4B.

The system 300 advantageously utilizes a plurality of reference structures 301-318 located on the head 320 of a screw 322 for providing positional information. The positioning information is particularly suitable for assisting precise placement of medical screws, such as pedicle screws, during a surgical procedure, and for facilitating return to a screw previously installed using the system.

In a preferred embodiment, the reference structures 301-318 represent a plurality of sets of reference structures, with the reference structures of each set being arranged in a common plane, but with each set of reference structures being located in distinct but parallel planes. For example, as seen in FIG. 13, a first set has reference structures 301 and 304 which lie in a common plane, a second set has reference structures 307 and 310 which lie in a common plane, and a third set has reference structures 313 and 316 which lie in a common plane. As will further be observed, the first, second, and third sets of reference structures are located in distinct, non-coplanar, but parallel planes.

The reference structures 301-318 preferably incorporate transmitter devices, such as transmitter devices 301a, 307a, and 318a, which may be referenced via receiver devices located on a corresponding driver 324 to provide positional information to a computerized control system to facilitate precise placement or other tracking of the location of the screw 322 in a patient. For example, circuitry 326 is associated with receivers on the driver 324, e.g., receivers 301b, 307b, 313b and the like. The circuitry 326 preferably communicates with an external computer controller 328 for receiving signals generated by the sensors. In this regard, the circuitry 326 may be directly connected to the computer controller 328 as by wiring, or may communicate as by wireless communication structure such as radio frequency and the like. Likewise, the transmitters and receivers may each be configured as transceivers.

The positional information may be input into the computer controller 328 and utilized, for example, to provide depth information based on the position of one or more of the reference structures and the degree of rotation of the screw as a function of the thread pitch. Also, the position information may be maintained in a database to facilitate returning to the site of the fastener, as may be desired for some medical procedures, or for subsequent removal of the screw. In this regard, the positioning system is believed to be particularly useful to facilitate docking of a medical driver to a previously installed medical fastener or other workpiece for subsequent medical procedures or removal thereof. This is particularly advantageous when attempting to return to previously implanted fasteners which have been in the patient for an extended period of time and have become overgrown with tissue. In such situations, the stored positional information may facilitate initial location of the previously installed device. This information may be used in conjunction with the relative positional information of the driver and the workpiece generated during a re-docking procedure to further facilitate re-docking of the driver on the head of the previously installed workpiece.

As will be appreciated, the positioning system may be suitable for broad application for use with positioning a variety of implantable medical devices or other workpieces. In this regard, the positioning system is particularly suitable for facilitating installation of artificial spinal discs. For example, as described previously, workpieces in the nature of spinal discs configured to include a castellated head and internal wireless communication devices are particularly advantageous in that they may be more precisely placed as compared to conventional spinal discs. However, it will be appreciated that such workpieces may further be configured to incorporate the structures described in connection with the positioning system, with the reference structures located on the head of the spinal disc or other workpiece to further facilitate initial positioning of the workpiece or re-docking of a driver or other holder device for removal or adjustment of the workpiece.

The screw 322 and the driver 324 preferably utilize a male/female castellated configuration of the type incorporated into the screws and drivers available from Uni-Screw Worldwide, Inc., of Knoxville, Tenn., and described in U.S. Patent Application Publication No. 2003/0209113, entitled “Integrated Fastening System,” and published Nov. 13, 2003, incorporated herein by reference. Thus, as shown in FIG. 14, the screw head 320 provides a female structure and has surfaces thereon which define the reference structures 301-318. As will be noted from FIG. 12, the driver 324 provides a male structure and includes corresponding surfaces thereon, such that a corresponding surface of the driver 324 bears against each of the marks 301-318 when the driver is mated with the head of the screw.

Each of the surfaces that defines the reference structures 301-318 preferably includes a discrete transmitter applied thereto, with the transmitter preferably being a thin film sensor configured as a light emitting diode to transmit light or as a radio frequency transmitter. For example, thin film transmitters 301a, 307a, and 313a are applied to the surfaces of the screw 322 corresponding to the reference surfaces 301, 307, and 313. Likewise, additional transmitters are preferably provided for the other reference structures.

In a similar manner, corresponding receivers, such as thin film sensors configured as light detectors or radio frequency receivers are correspondingly applied to the surfaces of the driver. For example, receivers 301b, 307b, and 313b are applied to the corresponding surfaces of the driver 324 for placement proximate the transmitters 301a, 307a, and 313a, respectively, when the tip of the driver 324 is inserted into the head.

As will be appreciated, when the transmitters associated with the screw are proximate the receivers associated with the driver, such as when the driver is mated with the screw, a circuit is completed to provide electrical signals corresponding to rotation of the screw head, position, and the like. As described, this information may be utilized by the computer controller to provide positional information relative to the head of the screw. This information may be utilized to precisely alter the depth of the screw. For example, if it is desired to back the screw out 0.2 mm, the computer algorithm would indicate that for the pitch of the screw, that the head should be rotated counter-clockwise 24 degrees. Accordingly, by having a plurality of sensors located as described, precise incremental rotational information concerning the head of the screw may be provided.

It will further be understood that the receivers and the transmitters may be sufficiently proximate to accomplish communication therebetween without the driver actually being mated with the screw head. Likewise, a receiver device having receiving elements corresponding to the receivers described herein may be provided remote from the screw to receive signals that may be utilized to guide the driver to the screw such as during a surgical procedure for removal of a previously implanted screw.

The positions of the receivers and the transmitters may also be reversed, with the receivers on the screw and the transmitters on the driver. Likewise, the driver and the screw head may be of reverse configuration, such that the driver represents the female structure and the screw head represents the male structure.

In addition, as noted previously, the system 300 may preferably be incorporated into the various embodiments described herein in connection with FIGS. 1-11. In this regard, it will be understood that the screw 322 and driver 324 may be configured to have the communicating bores and the associated structures as provided in the FIGS. 1-11, or that the structures described in connection with FIGS. 12-14 may be incorporated into the structures of FIGS. 1-11.

The foregoing description of certain exemplary embodiments of the present invention has been provided for purposes of illustration only, and it is understood that numerous modifications or alterations may be made in and to the illustrated embodiments without departing from the spirit and scope of the invention as defined in the following claims.

1. A medical fastener installation system for installing a medical fastener within a patient which system enables detection of electrically responsive tissue proximate the fastener during installation of the fastener, comprising (a) a fastener and (b) a driver engageable with the fastener for installing the fastener within a patient and disengageable from the fastener after installation of the fastener, wherein the driver and fastener are each configured to have communicating bores extending through their lengths for passage of (c) an electrically communicative element having a first portion located within the bore of the fastener, and in electrical communication with the exterior of the fastener and a second portion located within the bore of the driver and configured to be in electrical communication with the first portion of the electrically communicative element when the driver is engaged with the fastener and not in electrical communication with the first portion of the electrically communicative element when the driver is disengaged from the fastener, (d) a source of electrical power, and (e) a detector associated with the driver and configured to indicate the presence of electrically responsive tissue proximate the fastener during installation of the fastener with the driver when the driver and the fastener are engaged and the first and second portions of the electrically communicative element are placed in electrical communication with one another, the source of electrical power and the detector each being in electrical communication with the electrically conductive element when the driver and the fastener are engaged during installation of the fastener, wherein when an electrical force is applied to the electrically communicative element via the source of electrical power during installation of the fastener using the driver, electrical signals will be generated by the patient in response to the presence of electrically responsive tissue proximate the fastener, with such electrical signals being conducted via the engaged driver and fastener and the electrically communicative element to the detector to indicate the presence of electrically responsive tissue proximate the fastener to enable detection of the electrically responsive tissue during installation of the fastener.

2. The system of claim 1, wherein the fastener comprises a screw.

3. The system of claim 1, wherein the fastener comprises a screw having a castellated head and the driver has a tip configured to interfit with the castellated head of the screw.

4. The system of claim 1, wherein the electrically conductive element comprises an electrode wire.

5. (canceled)

6. The system of claim 1, wherein the electrically communicative element comprises a plurality of electrically conductive elements electrically coupled together.

7. The system of claim 1, wherein the fastener is made of a non-electrically conductive material.

8. The system of claim 1, wherein the fastener is made of an electrically conductive material.

9. The system of claim 1, wherein the source of electrical power applies electrical force in pulses.

10. The system of claim 1, wherein the source of electrical power is a direct current source.

11. The system of claim 1, wherein the source of electrical power is an alternating current source.

12. The system of claim 1, wherein the detector comprises an audible alarm.

13. The system of claim 1, wherein the detector comprises a light.

14. The system of claim 1, wherein the communicating bores define passages for introduction of a medicament to a patient, and wherein the system further includes a seal operatively associated with an inlet end of the bore of the fastener for selectively sealing and unsealing the inlet.

15. The system of claim 1, wherein the fastener includes a head defining a plurality of reference structures located thereon and oriented to define a plurality of sets of the reference structures, with the reference structures of each set being arranged in a common plane, but with each set of reference structures being located in distinct but parallel planes; the driver including a plurality of drive surfaces located and configured to abut the reference structures when the driver and the head are mated, with a plurality of first sensors being located on the reference structures and a plurality of second sensors being located on the drive surfaces, with each of the first sensors being operable with one of the second sensors to enable electrical signals to be generated corresponding to signals transmitted and received therebetween; and a computer control circuit in electrical communication with one of the first or second set of sensors for receiving the generated electrical signals.

16. A medical fastener installation system for installing a medical fastener within a patient which system enables detection of electrically responsive tissue proximate the fastener during installation of the fastener, comprising (a) a fastener and (b) a driver engageable with the fastener for installing the fastener within a patient and disengageable from the fastener after installation of the fastener, wherein the driver and fastener are each configured to have communicating bores for passage of (c) a tissue stimulating device having a first portion located within the bore of the fastener and in communication with the exterior of the fastener and a second portion located within the bore of the driver and configured to be in electrical communication with the first portion of the electrically communicative element when the driver is engaged with the fastener and not in electrical communication with the first portion of the electrically communicative element when the driver is disengaged from the fastener, (d) a source of stimulating power and (e) a detector associated with the driver and configured to indicate the presence of electrically responsive tissue proximate the fastener during installation of the fastener with the driver when the driver and fastener are engaged and the first and second portions of the electrically communicative element are placed in electrical communication with one another, the source of electrical power and the detector each being in communication with the tissue stimulating device, wherein when a stimulating force is applied to the stimulating device during installation of the fastener with the driver, signals will be generated by the patient in response to the presence of electrically responsive tissue proximate the fastener, with such signals being communicated to the detector to indicate that electrically responsive tissue is proximate the fastener to enable detection of the electrically responsive tissue during installation of the fastener.

17. The system of claim 16, wherein the communicating bores extend through the lengths of the driver and fastener.

18. The system of claim 16, wherein the communicating bore of the fastener comprises a blind bore and the stimulating device comprises a pair of wires, each in electrical communication with one of a pair of contacts located within the blind bore and extending through a sidewall of the fastener to an exterior portion thereof.

19. The system of claim 16, wherein the fastener comprises a screw.

20. The system of claim 16, wherein the fastener comprises a screw having a castellated head and the driver has a tip configured to interfit with the castellated head of the screw.

21. The system of claim 16, wherein the stimulating device comprises an electrode wire.

22. (canceled)

23. The system of claim 16, wherein the stimulating device comprises a plurality of electrically conductive elements electrically coupled together.

24. The system of claim 16, wherein the fastener is made of a non-electrically conductive material.

25. The system of claim 16, wherein the fastener is made of an electrically conductive material.

26. The system of claim 16, wherein the source of electrical power applies electrical force in pulses.

27. The system of claim 16, wherein the source of electrical power is a direct current source.

28. The system of claim 16, wherein the source of electrical power is an alternating current source.

29. The system of claim 16, wherein the detector comprises an audible alarm.

30. The system of claim 16, wherein the detector comprises a light.

31. The system of claim 16, wherein the communicating bores define passages for introduction of a medicament to a patient, and wherein the system further includes a seal operatively associated with an inlet end of the bore of the fastener for selectively sealing and unsealing the inlet.

32. The system of claim 16, wherein fastener includes a head defining a plurality of reference structures located thereon and oriented to define a plurality of sets of the reference structures, with the reference structures of each set being arranged in a common plane, but with each set of reference structures being located in distinct but parallel planes; the driver including a plurality of drive surfaces located and configured to abut the reference structures when the driver and the head are mated, with a plurality of first sensors being located on the reference structures and a plurality of second sensors being located on the drive surfaces, with each of the first sensors being operable with one of the second sensors to enable electrical signals to be generated corresponding to signals transmitted and received therebetween; and a computer control circuit in electrical communication with one of the first or second set of sensors for receiving the generated electrical signals.

33-96. (canceled)
